(19) United States
(12) Patent Application Publication
(10) Pub. No.: US 2012/0109114 A1
(43) Pub. Date: May 3, 2012

(54) HAIR REMOVAL BY PHOTOTHERMOLYSIS

(76) Inventors: Amir Waldman, Yarqona (IL); Gabi Lavi, Raanana (IL)

(21) Appl. No.: 12/912,978

(22) Filed: Oct. 27, 2010

(51) Int. Cl. A61B 18/18

(52) U.S. Cl. 606/3; 606/9

(57) ABSTRACT

Apparatus for photothermolysis including a removable and disposable cartridge mounted in a hand-held housing, the cartridge including a light source connectable to a power supply external to the cartridge, a reflector arranged with respect to the light source to direct light emanating from the light source towards skin via an opening formed in the cartridge to cause removal of hair on the skin by photothermolysis, and a spectral filter for filtering the light before the light exits the opening.

### FIELD OF THE INVENTION

The present invention relates to photothermolysis, such as methods and devices for treatment of skin and for skin hair permanent removal.

### BACKGROUND OF THE INVENTION

Treatment of the upper layer of the skin, epidermis and dermis is performed in order to achieve younger and nice appearance of the skin. Light therapy is a very effective tool in addressing a variety of lesions in the skin such as pigmented and vascular lesions, and hair follicles. Both laser light and incoherent light energy have been suggested for use, wherein pulsed optical energy heats the selected lesion without damaging the surrounding tissue. In order to selectively heat the lesion, the optical spectrum, pulse duration, energy density, and spot size have to be carefully set. Existing systems use pulse durations in the range of 2-100 msec and energy density of 10-60 J/cm².

Removal of excess or otherwise undesired hair from different parts of the human body has become more and more popular, particularly for cosmetic and aesthetic reasons.

### SUMMARY OF THE INVENTION

It is an object of the invention to provide a photothermolysis method that will allow the use of simple, small size and less expensive equipment. Methods and devices are described for treatment of skin and for skin hair permanent removal by electromagnetic irradiation applied to the hair follicles, with low volume and price. The methods may include using broad spectrum incoherent light and applying it in to the skin in a short pulse. The system may comprise a lamp housing pulse forming network. The invention may be used in a variety of dermatological applications, such as but not limited to, epilation, treatment of superficial vascular lesions, benign pigmented lesions, acne or bromidrosis, and for removal of unwanted hair, for example.

In the present invention, optimal pulse duration may result in a combined acoustical-thermal effect. As a result, destruction of the lesion is not totally based on heating but also gets some mechanical damage due to the acoustic wave that is generated by the short optical pulse. As a result of the combined acoustical-thermal effect, the lesion distraction occurs at much lower energies, e.g., 2-6 J/cm². Therefore, the device itself becomes much smaller and cheaper to build.

The apparatus of the present invention may be domestically used by all patients with no prior professional/training.

There is thus provided in accordance with an embodiment of the present invention apparatus for photothermolysis including a removable and disposable cartridge mounted in a hand-held housing, the cartridge including a light source connectable to a power supply external to the cartridge, a reflector arranged with respect to the light source to direct light emanating from the light source towards skin via an opening formed in the cartridge to cause removal of hair on the skin by photothermolysis, and a spectral filter for filtering the light before the light exits the opening.

In accordance with an embodiment of the present invention the cartridge includes an identification module that communicates with control electronics disposed in the hand-held housing, wherein the identification module permits operation of the apparatus only if authorized identification is established between the identification module and the control electronics. The communication between the identification module and the control electronics may be encrypted.

In accordance with an embodiment of the present invention the identification module together with the control electronics controls operating parameters of the apparatus, the operating parameters including at least one of number of flashes of the light, wavelength of the light, duration of the flashes of light and energy fluence of the light.

The light emanating from the light source towards skin may include, for example, and without limitation, flashes of incoherent light of wavelength of about 350-1200 nm for a duration of about 20-500 μsec and an energy fluence of about 2-6 J/cm². The light source may be a xenon lamp, for example. The light emanating from the light source towards skin may include a single pulse at one given place on the skin.

In accordance with an embodiment of the present invention the apparatus further includes a skin color sensor disposed in the hand-held housing for sensing color of the skin. The skin color sensor may include an optic sensor that senses color of light reflected from the skin back to the skin color sensor. Alternatively, the skin color sensor may include a thermal sensor that senses temperature of thermal energy reflected from the skin back to the skin color sensor.

A cooling system may be disposed inside the hand-held housing, such as a cooling fan.

### BRIEF DESCRIPTION OF THE DRAWINGS

These and additional constructional features and advantages of the invention will be more readily understood in the light of the ensuing description of embodiments thereof, given by way of example only, with reference to the accompanying drawings wherein:

FIG. 1 is a simplified partially cutaway pictorial illustration of apparatus for photothermolysis, constructed and operative in accordance with an embodiment of the present invention; and

FIGS. 2 and 3 are simplified pictorial and partially cutaway illustrations, respectively, of the apparatus of FIG. 1 connected to a control and power supply unit, in accordance with an embodiment of the present invention.

### DETAILED DESCRIPTION OF EMBODIMENTS

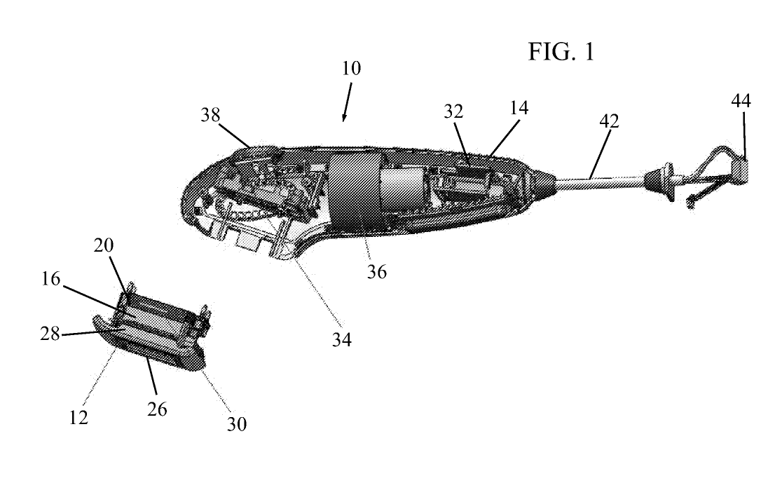

Reference is now made to FIG. 1, which illustrates apparatus 10 for photothermolysis, constructed and operative in accordance with a non-limiting embodiment of the present invention.

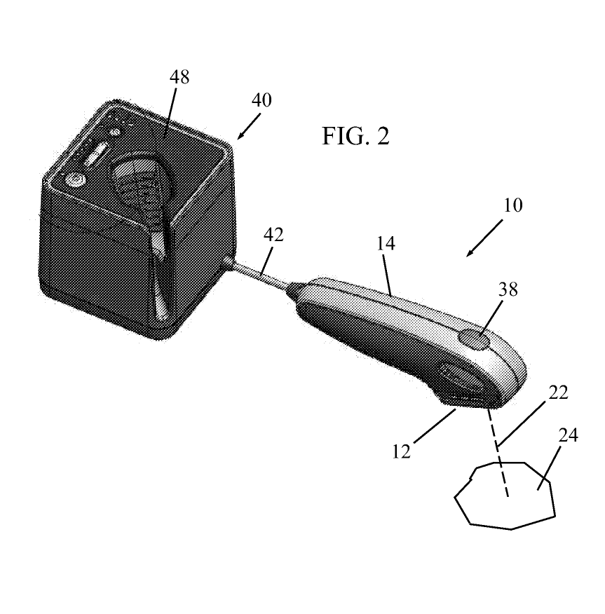

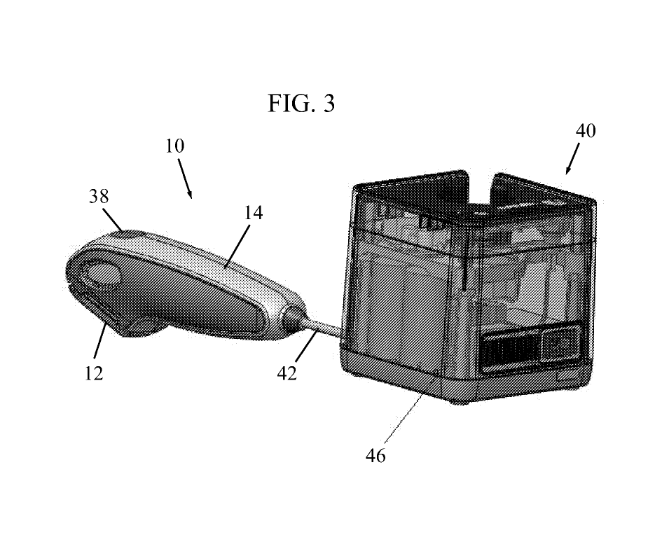

Apparatus 10 includes a removable and disposable cartridge 12 mounted in a hand-held housing 14. Hand-held housing 14 may be made of injection-molded plastic, for example. Cartridge 12 includes a light source 16 connectable to a power supply (external to cartridge 12, one example of which is shown in FIG. 3). Cartridge 12 also includes a reflector 20 arranged with respect to light source 16 to direct light 22 emanating from light source 16 towards skin 24 (seen in FIG. 2) via an opening 26 formed in cartridge 12. Reflector 20 can be selected to reflect and/or diffuse light emanating from light source 16, depending on the application. The light 22 causes removal of hair on the skin by photothermolysis. Cartridge 12 also includes a spectral filter 28 for filtering light 22 before exiting opening 26. Filter 28 may be, without limitation, a neutral density filter or a low cutoff filter in the visible and mostly ultraviolet portions of the spectrum.

The light 22 emanating from light source 16 may include, for example, and without limitation, flashes of incoherent light of wavelength of about 350-1200 nm for a duration of about 20-500 μsec and an energy fluence of about 2-6 J/cm². The light may be delivered as a single pulse at one place on the skin or a series of pulses at one place on the skin before moving to other treatment sites on the skin.

Light source 16 may be an incoherent light source, such as but not limited to, a xenon lamp or a gas-filled linear flash lamp, and may be either a single flash lamp or a plurality of flash lamps connected electrically in series to increase the energy fluence. The flash lamp can be linear, circular or helical, or any other shape. The flash lamp may have an outer enclosure made of bore silica, fuse silica or quartz.

In accordance with an embodiment of the present invention, cartridge 12 includes an identification module 30, such as an RFID (radio frequency identification) module, that communicates with control electronics 32 disposed in hand-held housing 14. The identification module 30 permits operation of apparatus 10 only if authorized identification is established between identification module 30 and control electronics 32. The communication between identification module 30 and control electronics 32 may be encrypted.

In accordance with an embodiment of the present invention, identification module 30 together with control electronics 32 controls operating parameters of apparatus 10, such as but not limited to, number of flashes of the light, wavelength of the light, duration of the flashes of light and/or energy fluence of the light. Control electronics 32 use well known components to communicate with identification module 30 and to control these parameters, and needs no detailed explanation for the skilled artisan.

In accordance with an embodiment of the present invention apparatus 10 further includes a skin color sensor 34 disposed in hand-held housing 14 for sensing color of the skin. (Alternatively, skin color sensor 34 may be mounted in cartridge 12.) Skin color sensor 34 may include an optic sensor that senses color of light reflected from the skin back to the skin color sensor. Alternatively, skin color sensor 34 may include a thermal sensor that senses temperature of thermal energy reflected from the skin by radiation heat transfer (and/or convection heat transfer or other modes of heat transfer) back to skin color sensor 34. (Skin color sensor 34 is arranged with respect to cartridge 12 so that cartridge 12 does not block reflected light or thermal energy to sensor 34.)

A cooling system 36 may be disposed inside hand-held housing 14, such as a cooling fan. Hand-held housing 14 may be provided with an operation switch 38.

Reference is now made to FIGS. 2 and 3. In accordance with an embodiment of the invention, the hand-held unit may be connected to a control and power supply unit 40, such as by means of electrical wiring conduit 42 and electrical connector 44 (which mates with a corresponding socket or connector in unit 40). Control and power supply unit 40 may include various control and power supply circuitry 46 known in the art of controls and power supplies. For example, the circuitry 46 may include a DC power supply, such as a linear or switching power supply, a power supply printed circuit board, a switch (e.g., SCR [semiconductor-controlled rectifier] switch), a transformer and one or more capacitors used to build the high voltage ignition to the flash lamp. The invention can be carried out with serial or parallel ignition. Unit 40 may supply its own power (such as one or more batteries) or may be connected to an external power source (such as a mains outlet) by a suitable electrical connection.

Control and power supply unit 40 may include a control panel 48 with various control buttons and displays.

To treat the skin, the required light fluence, peak power, pulse width and spectrum wavelength on the skin is delivered from the components of cartridge 12 on the skin.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Apparatus for photothermolysis comprising:
a removable and disposable cartridge mounted in a hand-held housing, said cartridge comprising a light source connectable to a power supply external to said cartridge, a reflector arranged with respect to said light source to direct light emanating from said light source towards skin via an opening formed in said cartridge to cause removal of hair on said skin by photothermolysis, and a spectral filter for filtering the light before the light exits said opening.

2. The apparatus according to claim 1, wherein said cartridge comprises an identification module that communicates with control electronics disposed in said hand-held housing, wherein said identification module permits operation of said apparatus only if authorized identification is established between said identification module and said control electronics.

3. The apparatus according to claim 2, wherein communication between said identification module and said control electronics is encrypted.

4. The apparatus according to claim 2, wherein said identification module together with said control electronics controls operating parameters of said apparatus, the operating parameters comprising at least one of number of flashes of the light, wavelength of the light, duration of the flashes of light and energy fluence of the light.

5. The apparatus according to claim 1, wherein the light emanating from said light source towards skin comprises flashes of incoherent light of wavelength of about 350-1200 nm for a duration of about 20-500 μsec and an energy fluence of about 2-6 J/cm².

6. The apparatus according to claim 1, wherein the light source is a xenon lamp.

7. The apparatus according to claim 1, wherein the light emanating from said light source towards skin comprises a single pulse at one given place on the skin.

8. The apparatus according to claim 1, further comprising a skin color sensor disposed in said hand-held housing for sensing color of the skin.

9. The apparatus according to claim 8, wherein said skin color sensor comprises an optic sensor that senses color of light reflected from the skin back to the skin color sensor.

10. The apparatus according to claim 8, wherein said skin color sensor comprises a thermal sensor that senses temperature of thermal energy reflected from the skin back to the skin color sensor.

11. The apparatus according to claim 1, further comprising a cooling system inside said hand-held housing.

12. The apparatus according to claim 11, wherein said cooling system comprises a cooling fan.
